(19) United States
(12) Patent Application Publication
(10) Pub. No.: US 2020/0253788 A1
(43) Pub. Date: Aug. 13, 2020

(54) CONTOURED FOAM DRESSING SHAPED FOR PROVIDING NEGATIVE PRESSURE TO INCISIONS IN THE SHOULDER

(71) Applicant: KCI Licensing, Inc., San Antonio, TX (US)

(72) Inventors: Jonathan G. Rehbein, San Antonio, TX (US); Richard M. Kazala, San Antonio, TX (US); Larry Tab Randolph, San Antonio, TX (US); Luke A. Perkins, San Antonio, TX (US)

(21) Appl. No.: 16/780,387

(22) Filed: Feb. 3, 2020

Related U.S. Application Data
(60) Provisional application No. 62/802,541, filed on Feb. 7, 2019.

(51) Int. Cl. A61F 13/14; A61M 1/00; A61F 5/058; B29C 44/04; B29C 44/42; B29C 44/56

(52) U.S. Cl. CPC A61F 13/146; A61M 1/0088; A61F 5/05858; A61M 1/0086; A61F 2013/0028; B29C 44/42; B29C 44/56; A61M 2210/083; B29C 44/04

(57) ABSTRACT

A negative pressure wound treatment (NPWT) dressing system for treating shoulder incisions. The NPWT dressing system includes a wound dressing, an immobilization device configured to immobilize a shoulder of a patient, and a negative pressure source coupled to the immobilization device. The wound dressing includes a drape layer, a manifold layer, and a reduced-pressure interface integrated with the drape layer and the negative pressure source. The drape layer has a first surface and a second, wound-facing, surface. The drape layer is substantially impermeable to liquid and substantially permeable to vapor. The manifold layer has a first surface and a second, wound-facing surface. The manifold layer has a first lobe, a second lobe substantially aligned with the first lobe, and a third lobe extending substantially perpendicular to the first lobe and the second lobe.

### CROSS-REFERENCE TO RELATED APPLICATIONS

This application claims the benefit of priority to U.S. Provisional Application No. 62/802,541, filed on Feb. 7, 2019, which is incorporated herein by reference in its entirety.

### BACKGROUND

The present disclosure relates generally to a wound therapy system, and more particularly to a wound therapy system configured to provide negative pressure wound therapy to the site of one or more shoulder incisions.

Negative pressure wound therapy (NPWT) is a type of wound therapy that involves applying a negative pressure to a wound treatment area to promote wound healing. NPWT can be used to treat wounds in the shoulder area caused by arthroscopic shoulder surgeries. Recent developments in NPWT therapy include the use of adhesive wound dressings that can be positioned over a wound to treat the wound and the surrounding area. However, existing adhesive NPWT dressings are primarily linear dressings designed to treat linear wounds. In most instances, shoulder surgeries involve three incisions arranged in a non-linear configuration. A first incision is at a front portion of a patient's shoulder, a second incision is at a back portion of the patient's shoulder, and a third incision is at a top portion of the patient's arm proximate the patient's shoulder. Existing NPWT dressings are configured to treat linear incisions and can be time-consuming to modify to treat the specific incision pattern used in arthroscopic shoulder surgeries.

### SUMMARY

One implementation of the present disclosure is a negative pressure wound treatment (NPWT) dressing system for use with shoulder incisions. The wound dressing system includes a wound dressing, an immobilization device, and a negative pressure source. The wound dressing includes a drape layer, a manifold layer, and a reduced pressure interface. The drape layer has a first surface and a second, wound-facing, surface. The drape layer is substantially impermeable to liquid and substantially permeable to vapor. The manifold layer has a first surface and a second, wound-facing surface. The manifold layer has a first lobe, a second lobe substantially aligned with the first lobe, and a third lobe extending substantially perpendicular to the first lobe and the second lobe. The reduced-pressure interface is integrated with the drape layer. The immobilization device is configured to immobilize a shoulder of a patient. The negative pressure source is in fluid communication with the reduced pressure interface. The negative pressure source is coupled to the immobilization device.

Another implementation of the present disclosure is a negative pressure wound therapy (NPWT) dressing. The NPWT dressing includes a drape layer, a manifold layer, a base layer, and a reduced pressure interface. The drape layer has a first surface and a second, wound-facing, surface. The drape layer is substantially impermeable to liquid and substantially permeable to vapor. The manifold layer has a first surface and a second, wound-facing surface. The manifold layer has a first lobe, a second lobe generally aligned with the first lobe, and a third lobe extending substantially perpendicular to the first lobe and the second lobe. The base layer is configured to secure the drape layer to the manifold layer. The base layer is configured to secure the wound dressing to a patient's tissue. The reduced-pressure interface is integrated with the drape layer.

Another implementation of the present disclosure is a negative pressure wound treatment (NPWT) dressing system for use with shoulder incisions. The wound dressing system includes a wound dressing, an immobilization device, and a negative pressure source. The wound dressing includes a manifold layer, an adhesive layer, and a reduced pressure interface. The manifold layer defines a substantially elbow-shaped channel having a first portion configured to receive an upper portion of a shoulder of a patient and a second portion angled relative to the first potion and configured to receive an upper portion an arm of the patient. The manifold layer has a first surface that is substantially impermeable to fluid and a second, wound-facing surface that is substantially permeable to fluid. The adhesive layer is coupled along a perimeter of the second surface of the manifold layer and configured to secure the wound dressing to the patient's tissue. The reduced pressure interface is integrated with the first surface of the manifold layer. The immobilization device is configured to immobilize the shoulder. The negative pressure source is in fluid communication with the reduced pressure device. The negative pressure source is coupled to the immobilization device.

Another implementation of the present disclosure is a method of forming a three-dimensional wound-dressing shaped to receive a shoulder. The method includes injection-molding a foam into a mold defining a substantially elbow-shaped channel having a first surface and a second, wound-facing, surface that is substantially permeable to fluid. The method includes curing the foam such that an exterior layer of the foam is fluid-impermeable and an interior portion of the foam is porous. The method includes removing at least a portion of the exterior layer of the foam from the second surface substantially inward of a perimeter of the second surface. The method includes securing an adhesive layer to the perimeter of the second surface, the adhesive layer configured to secure the wound dressing to a patient's tissue. The method includes removing a portion of the exterior layer of the foam from the first surface. The method includes positioning a reduced-pressure interface over the removed portion of the exterior layer of the foam of the first surface.

Another implementation of the present disclosure is a method of deploying a negative pressure wound therapy (NPWT) dressing on a shoulder. The method includes immobilizing a shoulder of a patient relative to a body of the patient with an immobilization device. The method includes securing a wound dressing to a shoulder treatment area. The securing step includes securing a first lobe of a wound dressing manifold proximate a wound treatment area on a front of a shoulder of the patient, securing a second lobe of the wound dressing manifold proximate a wound treatment area on a back of the shoulder, and securing a third lobe of the wound dressing proximate a treatment area on a top portion of an arm of the patient. The first lobe is substantially aligned with the second lobe. The method includes coupling the negative pressure source to a reduced-pressure interface of the wound dressing. The method includes coupling the negative pressure source to the immobilization device.

Those skilled in the art will appreciate that the summary is illustrative only and is not intended to be in any way limiting. Other aspects, inventive features, and advantages of the devices and/or processes described herein, as defined solely by the claims, will become apparent in the detailed description set forth herein and taken in conjunction with the accompanying drawings.

### BRIEF DESCRIPTION OF THE DRAWINGS

FIG. 1 illustrates a perspective view of a shoulder area of a representative patient undergoing shoulder surgery.

FIG. 2 is a front view of a wound dressing according to an exemplary embodiment.

FIG. 3 is a perspective view of the wound dressing of FIG. 2 according to an exemplary embodiment.

FIG. 4 is an exploded view of the wound dressing of FIG. 2 according to an exemplary embodiment.

FIG. 5 is a perspective view of a manifold layer of the wound dressing of FIG. 2 according to an exemplary embodiment.

FIG. 6 is an exploded view of a manifold layer of the wound dressing of FIG. 2 according to another exemplary embodiment.

FIG. 7 is a perspective view of the wound dressing of FIG. 2 adhered to a representative person's shoulder.

FIG. 8 is a perspective view of a wound dressing according to another exemplary embodiment.

FIG. 9 is a section view of the wound dressing of FIG. 8 taken along lines 9-9 of FIG. 8.

FIG. 10 is a process diagram illustrating a method of manufacturing the wound dressing of FIG. 8 according to an exemplary embodiment.

FIG. 11 is a perspective view of a wound dressing adhered to a representative person's torso according to another exemplary embodiment.

FIG. 12 is a perspective view of a wound dressing adhered to a representative person's torso according to another exemplary embodiment.

FIG. 13 is a perspective view of a wound dressing adhered to a representative person's torso according to another exemplary embodiment.

FIG. 14 is a perspective view of the wound dressing of FIG. 13.

### DETAILED DESCRIPTION

### Overview

Referring generally to the FIGURES, a wound therapy system for treating wounds of curved body parts is shown, according to various embodiments. More specifically, the wound therapy system is for treating wounds in the shoulder area. The wound therapy system includes a wound dressing and a negative pressure wound therapy (NPWT) system. The phrase “negative pressure” means a pressure less than an ambient or atmospheric pressure. While the amount and nature of reduced pressure applied to the wound treatment area can vary according to the application, the reduced pressure typically is between −5 mm Hg and −500 mm Hg and more typically between −100 mm Hg and −300 mm Hg.

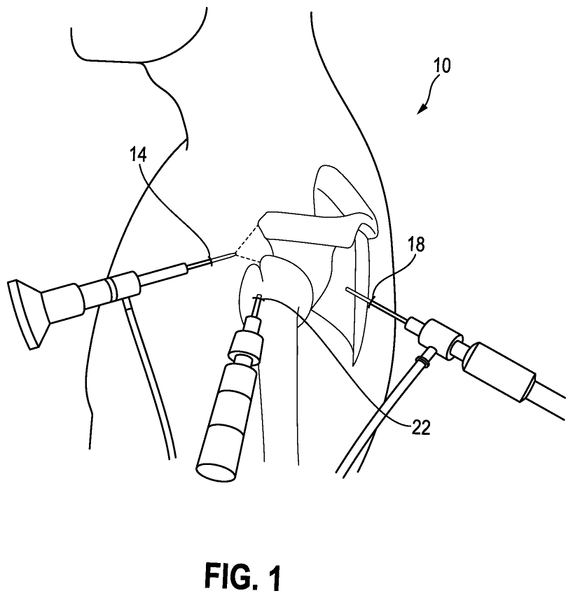

FIG. 1 illustrates an exemplary shoulder 10 of a patient undergoing arthroscopic shoulder surgery. Arthroscopic shoulder surgeries typically include a first incision 14 at front of an upper portion of a patient's shoulder, second incision 18 at a back of an upper portion of a patient's shoulder, and a third incision 22 at an upper portion of a patient's arm. The wound treatment area includes the first incision 14, healthy tissue surrounding the first incision 14, the second incision 18, healthy tissue surrounding the second incision 18, the third incision 22, and healthy tissue surrounding the second incision 18. The wound dressings described herein are configured to substantially cover the wound treatment area and apply NPWT to the wound treatment area. During arthroscopic shoulder surgery, several liters of fluid are pumped into the shoulder area. Applying NPWT to the entire wound treatment area can facilitate patient healing by lifting the healthy tissue surrounding the incisions, which facilitates absorption of the fluid by the lymphatic system of the patient's body. In some embodiments, the NPWT system can remove excess fluid that cannot be absorbed by the body. For example, fluid (wound exudate, fluid injected during surgery, etc.) can drain from the wound treatment area via the negative pressure conduit or a dedicated drain line. Fluid can be collected by a removed fluid reservoir of the NPWT system.

In some embodiments, the wound dressing is substantially T-shaped and includes a first lobe, a second lobe, and a third lobe. The first lobe and the second lobe are generally aligned and the third lobe is generally perpendicular to the first lobe and the second lobe. The first lobe and the second lobe are substantially half-ellipses and the third lobe is substantially circular. The first lobe and the second lobe are connected to the third lobe by a connection portion that is narrower than the first lobe, the second lobe, and the third lobe. The wound dressing is shaped to wrap around the shoulder of the patient. The first lobe is configured to overlie the first incision 14 and the healthy tissue surrounding the first incision 14 at the front portion of the patient's shoulder. The second lobe is configured to overlie the second incision 18 and the healthy tissue surrounding the second incision 18 at the back portion of the patient's shoulder. The third lobe is configured to overlie the third incision 22 and the healthy tissue surrounding the third incision 22 at the upper portion of the patient's arm. In some embodiments, the wound dressing includes a concave contour that is generally aligned with the third lobe. The concave contour is configured to prevent the wound dressing from overlying the patient's trapezius muscle. The shape of the wound dressing is generally symmetric to allow placement of the wound dressing on either the left or the right shoulder.

In some embodiments, the wound dressing has a substantially 3D-shape configured to conform to the shoulder wound treatment area. In such an embodiment, the wound dressing forms an elbow-shaped channel that includes a first portion and a second portion that is angled relative to the first portion. In some embodiments, the second portion is at a substantially obtuse angle relative to the first portion. The first portion is configured to overlie a first incision and the surrounding healthy tissue at a front portion of the patient's shoulder, a top portion of the patient's shoulder, and a second incision and the surrounding healthy tissue at a back portion of the patient's shoulder. In some embodiments, a first lobe and a second lobe extend from the first portion. In some embodiments, the first lobe and the second lobe are generally perpendicular to the first portion. The first lobe is configured to overlie the first incision and healthy tissue surrounding the first incision. The second lobe is configured to overlie the second incision and healthy tissue surrounding the first incision.

Generally, the wound dressing is used in conjunction with an immobilization device such as a sling or a belt that is configured to immobilize a patient's arm relative to the patient's torso to immobilize the patient's shoulder joint. A negative pressure source or pump and a removed fluid container are integrated into the immobilization device. The wound dressing includes a negative pressure interface that facilitates fluid communication between the wound dressing and a negative pressure conduit that is coupled the negative pressure source. A portion of the negative pressure conduit proximate the NPWT system is positioned within the immobilization device. Integration of the NPWT system with the immobilization device allows the patient to conveniently transport the negative pressure source while the patient is undergoing NPWT.

In some embodiments, the wound dressing is configured to drain excess fluid from the wound treatment area. In such embodiments, the removed-fluid container can be configured to store a fluid removed from the wound treatment area (e.g., wound exudate, fluid injected during surgery, etc.). In some embodiments, the removed fluid container is positioned upstream of the negative pressure source so that fluid can drain from the wound dressing via the negative pressure conduit and accumulate in the removed-fluid container. In other embodiments, the removed-fluid container can be fluidly coupled to the wound treatment area via a fluid removal line that is separate from the negative pressure conduit. The NPWT can help reduce the chance of the wounds developing seroma, scaring, infection, or other adverse complications.

Additional features and advantages of the wound therapy system are described in detail below.

### Wound Dressing

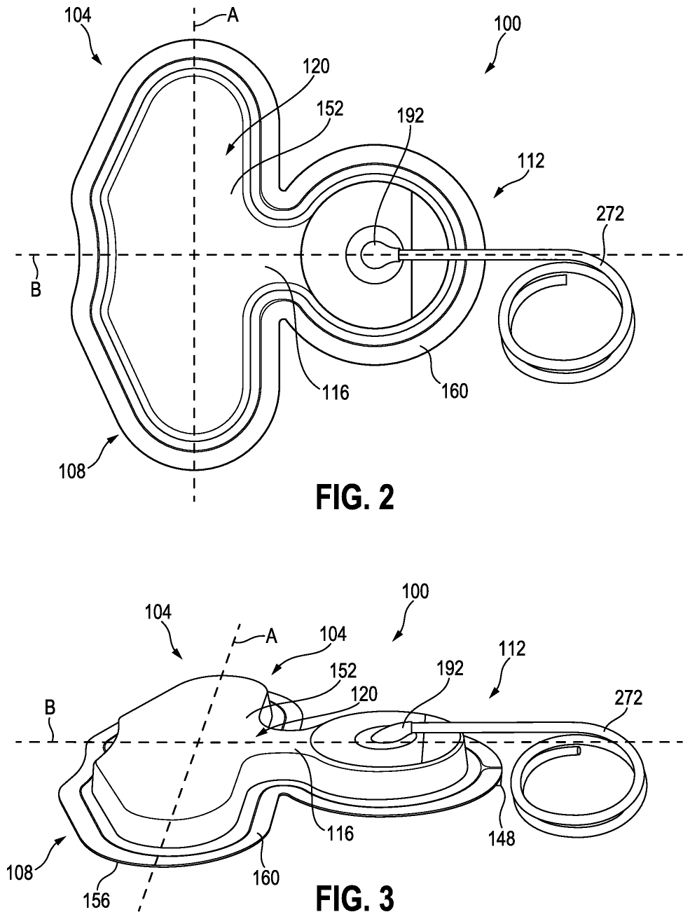

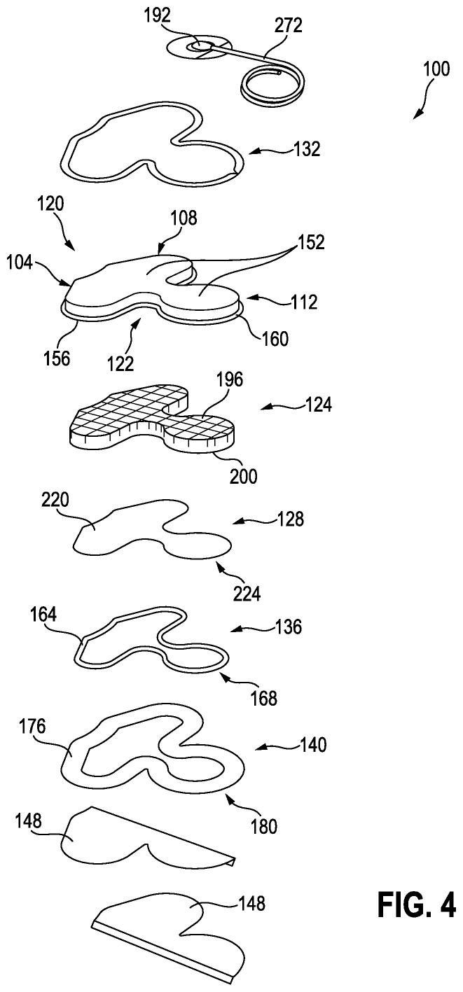

Referring now to FIGS. 2-4, a wound dressing 100 is shown, according to an exemplary embodiment. FIG. 2 is a front view of the wound dressing 100. FIG. 3 is a perspective view of the wound dressing 100. FIG. 4 is an exploded view illustrating several layers 120-148 of the wound dressing 100.

In various embodiments, the wound dressing 100 can be formed as a substantially flat sheet for topical application to wounds. The wound dressing 100 is generally planar, but can wrap around a shoulder of a patient to conform to the three-dimensional shape of a wound treatment area at the shoulder of the patient. The wound dressing 100 is substantially T-shaped and includes a first lobe 104, a second lobe 108, and a third lobe 112. The first lobe 104 and the second lobe 108 are substantially half-ellipses and are aligned along an axis A. The third lobe 112 is substantially circular and is connected to the first lobe 104 and the second lobe 108 by a connection portion 116 that is narrower than the third lobe 112. The third lobe 112 is substantially perpendicular to the first lobe 104 and the second lobe 108. The first lobe 104 is configured to overlie an incision at a front portion of a patient's shoulder and healthy tissue surrounding the incision. The second lobe 108 is configured to overlie an incision at a back portion of a patient's shoulder 10 and healthy tissue surrounding the incision. The third lobe 112 is configured to overlie an incision at an upper portion of a patient's arm and healthy tissue surrounding the incision. The wound dressing 100 is substantially symmetric about an axis B so that the wound dressing 100 can be deployed on a patient's right shoulder or a patient's left shoulder without requiring modification.

The wound dressing 100 is shown to include a plurality of layers, including a drape layer 120, a manifold layer 124, a wound-interface layer 128, a semi-rigid support layer 132, a first adhesive layer 136, and a second adhesive layer 140. In some embodiments, the wound dressing 100 includes a removable cover sheet 148 to cover the manifold layer 124, the wound-interface layer 128, and the second adhesive layer 140 before use.

### Drape Layer

The drape layer 120 is shown to include a first surface 152 and a second, wound-facing, surface 156 opposite the first surface 152. When the wound dressing 100 is applied to a wound, the first surface 152 faces away from the wound, whereas the second surface 156 faces toward the wound. The drape layer 120 supports the manifold layer 124 and the wound-interface layer 128 and provides a barrier to passage of microorganisms through the wound dressing 100. The drape layer 120 is configured to provide a sealed space over a wound or incision. In some embodiments, the drape layer 120 is an elastomeric material or may be any material that provides a fluid seal. “Fluid seal” means a seal adequate to hold pressure at a desired site given the particular reduced-pressure subsystem involved. The term “elastomeric” means having the properties of an elastomer and generally refers to a polymeric material that has rubber-like properties. Examples of elastomers may include, but are not limited to, natural rubbers, polyisoprene, styrene butadiene rubber, chloroprene rubber, polybutadiene, nitrile rubber, butyl rubber, ethylene propylene rubber, ethylene propylene diene monomer, chlorosulfonated polyethylene, polysulfide rubber, polyurethane, EVA film, co-polyester, and silicones. As non-limiting examples, the drape layer 120 may be formed from materials that include a silicone, 3M Tegaderm® drape material, acrylic drape material such as one available from Avery, or an incise drape material.

The drape layer 120 may be substantially impermeable to liquid and substantially permeable to water vapor. In other words, the drape layer 120 may be permeable to water vapor, but not permeable to liquid water or wound exudate. This increases the total fluid handling capacity (TFHC) of wound dressing 100 while promoting a moist wound environment. In some embodiments, the drape layer 120 is also impermeable to bacteria and other microorganisms. In some embodiments, the drape layer 120 is configured to wick moisture from the manifold layer 124 and distribute the moisture across the first surface 152.

As shown in FIG. 4, the drape layer 120 defines a cavity 122 for receiving the manifold layer 124, the wound-interface layer 128, and the first adhesive layer 136. The manifold layer 124, the wound-interface layer 128, and the first adhesive layer 136 can have a similar perimeter or profile. In some embodiments, a perimeter of the drape layer 120 extends beyond (e.g. circumscribes) the perimeter of the manifold layer 124 to provide a margin 160. The first adhesive layer 136 includes a first surface 164 and a second, wound-facing surface 168. Both first surface 164 and the second surface 168 are coated with an adhesive, such as an acrylic adhesive, a silicone adhesive, and/or other adhesives. The first surface 164 of the first adhesive layer 136 is secured to the second surface 172 of the wound-interface layer 128. The second surface 168 of the first adhesive layer 136 is secured to the second adhesive layer 140. The second adhesive layer 140 includes a first surface 176 and a second, wound-facing surface 180. The second surface 168 of the first adhesive layer 136 is secured to the first surface 176 of the second adhesive layer 140. The second surface 180 of the second adhesive layer 140 is coated with an acrylic adhesive, a silicone adhesive, and/or other adhesives. The adhesive applied to the second surface 180 of the second adhesive layer 140 is intended to ensure that the wound dressing 100 adheres to the surface of the patient's tissue and that the wound dressing 100 remains in place throughout the wear time. The second adhesive layer 140 has a perimeter or profile that is similar to a perimeter or profile of the margin 160. In the illustrated embodiment, the first surface 176 of the second adhesive layer 140 is welded to the margin 160. In other embodiments, the first surface 176 of the second adhesive layer is secured to the margin 160 using an adhesive, such as an acrylic adhesive, a silicone adhesive, or another type of adhesive. The margin 160 and/or the second adhesive layer 140 may extend around all sides of the manifold layer 124 such that the wound dressing 100 is a so-called island dressing. In other embodiments, the margin 160 and/or the second adhesive layer 140 can be eliminated and the wound dressing 100 can be adhered to the patient's tissue using other techniques. In some embodiments, the first adhesive layer 136, and the second adhesive layer 140 can collectively form a base layer that includes an adhesive on both sides that is (i) configured to secure the drape layer 120 to the manifold layer 124, the optional wound-interface layer 128, and (ii) configured to secure the wound dressing 100 to a patient's tissue. In some embodiments, the base layer can be integrally formed with the drape layer 120. In some embodiments, the base layer can be a layer of a polyurethane film having a first surface and a second, wound-facing surface. Both the first surface and the second surface can be coated with an adhesive (such as an acrylic or silicone adhesive). In some embodiments, the wound-facing surface of the base layer can include a hydrocolloid adhesive.

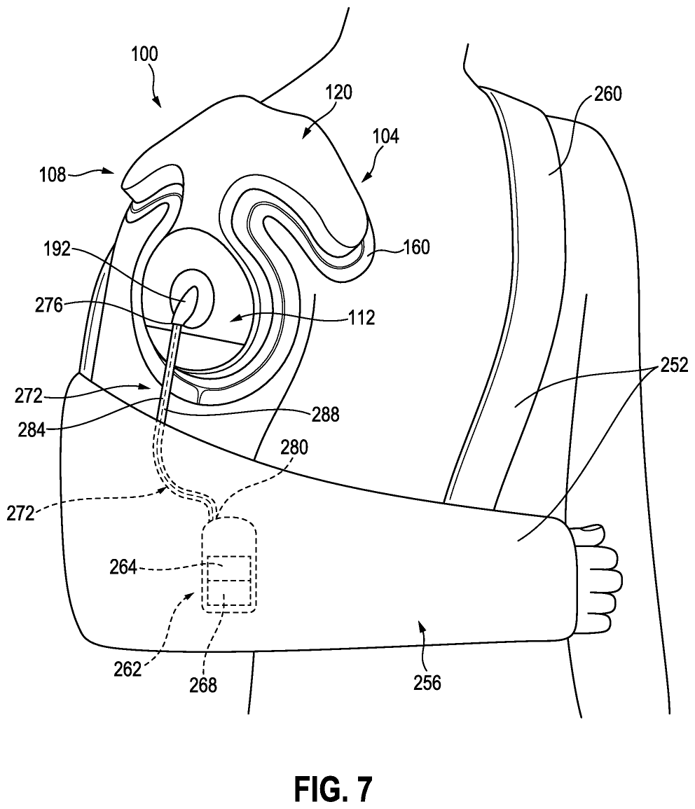

In some embodiments, a reduced-pressure interface 192 can be integrated with the drape layer 120. The reduced-pressure interface 192 can be in fluid communication with the negative pressure system through a negative pressure conduit 272. The reduced-pressure interface 192 is configured to allow fluid communication between a negative pressure source 268 (FIG. 7) and the wound dressing 100 (e.g., through the drape layer 120) via the negative pressure conduit 272 coupled between the reduced-pressure interface 192 and the negative pressure source 268 such that negative pressure generated by the negative pressure source 268 can be applied to the wound dressing 100 (e.g., through the drape layer 120). In some embodiments, the reduced-pressure interface 192 can be integrated (e.g., integrally formed) with the drape layer 120. In other embodiments, the reduced-pressure interface 192 can be separate from the drape layer 120 and configured to be coupled to the drape layer 120 by a user. In the illustrated embodiment, the reduced-pressure interface 192 is positioned above the third node 112. In other embodiments, the reduced-pressure interface 192 can be positioned elsewhere on the drape layer 120.

With continued reference to FIG. 4, the semi-rigid support layer 132 is positioned above the first surface 152 of the drape layer 120. The semi-rigid support layer 132 is spaced from but proximate the margin 160 and the second adhesive layer 140. The semi-rigid support layer 132 is made of a semi-rigid material and helps the wound dressing 100 maintain rigidity before the wound dressing 100 is secured to the surface of the patient. The semi-rigid support layer 132 is intended to be removed from the drape layer 120 after the wound dressing 100 has been secured to the patient's tissue.

In some embodiments, the second surface 156 of the drape layer 120 contacts the manifold layer 124. The second surface 156 of the drape layer 120 may be adhered to the manifold layer 124 or may simply contact the manifold layer 124 without the use of an adhesive.

In some embodiments, the adhesive applied to the second surface 156 of the drape layer 120 is moisture vapor transmitting and/or patterned to allow passage of water vapor therethrough. The adhesive may include a continuous moisture vapor transmitting, pressure-sensitive adhesive layer of the type conventionally used for island-type wound dressings (e.g. a polyurethane-based pressure sensitive adhesive).

### Manifold Layer

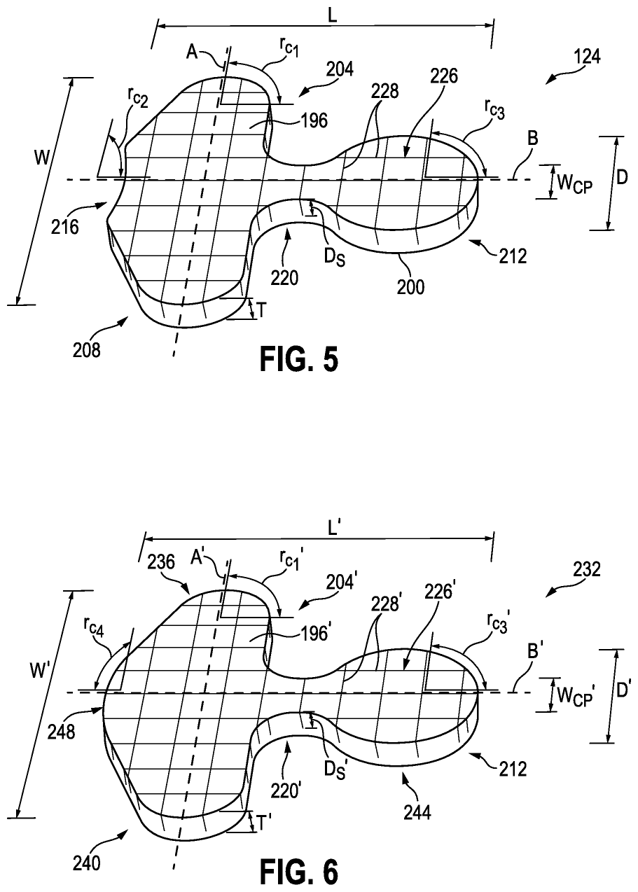

Referring to FIG. 5, the manifold layer 124 is shown to include a first surface 196 and a second, wound-facing surface 200 opposite the first surface 196. When the wound dressing 100 is applied to a wound, the first surface 196 faces away from the wound, whereas the second surface 200 faces toward the wound. In some embodiments, the first surface 196 of the manifold layer 124 contacts the second surface 156 of the drape layer 120. In some embodiments, the second surface 200 of the manifold layer 124 contacts the wound-interface layer 128. The manifold layer 124 is configured for transmission of negative pressure to the patient's tissue at and/or proximate a wound and/or incision. The manifold layer 124 is configured to wick fluid (e.g. exudate) from the wound and includes in-molded manifold layer structures for distributing negative pressure throughout the wound dressing 100 during negative pressure wound therapy treatments.

The manifold layer 124 can be made from a porous and permeable foam-like material and, more particularly, a reticulated, open-cell polyurethane or polyether foam that allows good permeability of wound fluids while under a reduced pressure. One such foam material that has been used is the V.A.C.® Granufoam™ material that is available from Kinetic Concepts, Inc. (KCI) of San Antonio, Tex. Any material or combination of materials might be used for the manifold layer 124 provided that the manifold layer 124 is operable to distribute the reduced pressure and provide a distributed compressive force along the wound treatment area.

The reticulated pores of the Granufoam™ material that are in the range from about 400 to 600 microns, are preferred, but other materials may be used. The density of the manifold layer material, e.g., Granufoam™ material, is typically in the range of about 1.3 lb/ft³-1.6 lb/ft³ (20.8 kg/m³-25.6 kg/m³). A material with a higher density (smaller pore size) than Granufoam™ material may be desirable in some situations. For example, the Granufoam™ material or similar material with a density greater than 1.6 lb/ft³ (25.6 kg/m³) may be used. As another example, the Granufoam™ material or similar material with a density greater than 2.0 lb/ft³ (32 kg/m³) or 5.0 lb/ft³ (80.1 kg/m³) or even more may be used. The more dense the material is, the higher compressive force that may be generated for a given reduced pressure. If a foam with a density less than the tissue at the tissue site is used as the manifold layer material, a lifting force may be developed. In one illustrative embodiment, a portion, e.g., the edges, of the wound dressing 100 may exert a compressive force while another portion, e.g., a central portion, may provide a lifting force.

The manifold layer material may be a reticulated foam that is later felted to thickness of about one third (⅓) of the foam's original thickness. Among the many possible manifold layer materials, the following may be used: Granufoam™ material or a Foamex® technical foam (www.foamex.com). In some instances, it may be desirable to add ionic silver to the foam in a microbonding process or to add other substances to the manifold layer material such as antimicrobial agents. The manifold layer material may be isotropic or anisotropic depending on the exact orientation of the compressive forces that are desired during the application of reduced pressure. The manifold layer material may also be a bio-absorbable material.

As shown in FIGS. 2-5, the manifold layer 124 is generally symmetrical about the axis B. The manifold layer 124 is substantially T-shaped and includes a first lobe 204, a second lobe 208, and a third lobe 212. The manifold layer 124 can have a length L ranging from approximately 7.44 inches to 11.16 inches. In some embodiments, the length L is approximately 9.3 inches. The manifold layer 124 can have a width W ranging from approximately 7.76 inches to approximately 11.64 inches. In some embodiments, the width W is approximately 9.7 inches. The manifold layer 124 can have a thickness T ranging from approximately 0.64 inches to 0.96 inches. In some embodiments, the thickness T is approximately 0.8 inches.

The first lobe 204 and the second lobe 208 are substantially elliptical. The first lobe 204 and the second lobe 208 can each have a radius of curvature rc₁ ranging from approximately 1.04 inches to 1.56 inches. In some embodiments, the first lobe 204 and the second lobe 208 can each have a radius of curvature of approximately 1.3 inches. The first lobe 204 and the second lobe are substantially aligned along the axis A. A concave portion 216 extends along a portion of the perimeter of the wound dressing 100 that is between the first lobe 204 and the second lobe 208. The concave portion 216 is substantially aligned with the third lobe 212 along the axis B. The concave portion 216 is positioned to prevent the wound dressing from overlying a trapezius muscle of a patient and/or contacting the patient's neck when the wound dressing 100 is secured to the patient's shoulder. The concave portion 216 can have a radius of curvature rc₂ ranging from approximately 4 inches to approximately 6 inches. In some embodiments, the radius of curvature rc₂ can be approximately 5 inches.

The third lobe 212 is substantially perpendicular to the first lobe 204 and the second lobe 208. The third lobe 212 is subsantially circular. The third lobe can have a radius of curvature rc₃ ranging from approximately 1.6 inches to approximately 2.4 inches. In some embodiments, the radius of curvature rc₃ can be approximately 2.0 inches. The third lobe 212 is connected to the first lobe 204 and the second lobe 208 by the connecting portion 220. The connecting portion 220 has a width W_{CP} smaller than a diameter D of the third lobe 212. The width W_{CP} of the connecting portion can range from approximately 1.2 inches to approximately 1.8 inches. In some embodiments, the width W_{CP} can be approximately 1.5 inches.

As is best shown in FIG. 5, a scoring pattern 226 is formed in the first surface 196 of the manifold layer 124. The scoring pattern 226 is shown for example as an arrangement of “slits” or scores (e.g., “mango-cuts”) formed in the manifold layer 124 (e.g. formed by laser-scoring or other suitable processes). More particularly, the scoring pattern 226 is cut into the first surface 196 of the manifold layer 124. In the embodiment of FIG. 5, the scoring pattern 226 extends between the first surface 196 and the second surface 200 but does not extend completely to the second surface 200. The scoring pattern 226 can have a depth D_{S} that can range from approximately 0.2 inches to 0.5 inches. In some embodiments, the depth D_{S} is approximately 0.28 inches. According to the illustrated embodiment, the scoring pattern 226 is a generally square pattern. However, in other embodiments, the scoring pattern 226 can be a different geometrical pattern. When the wound dressing 100 is used on a generally flat (e.g. two-dimensional) portion of the wound treatment area, such as for example a front of a shoulder or a back of a patient's shoulder, the scores 228 of the scoring pattern 226 are generally vertical and are in close proximity to adjacent scores 228 of the scoring pattern 226. In instances when the wound dressing 100 is secured to a curved (e.g. three-dimensional) surface, such as a transition portion of the surface that extends between the front of the shoulder and the top of the shoulder, the back of the shoulder and the top of the shoulder, and the shoulder and the top of the arm, the scores 228 of the scoring pattern 226 splay apart to facilitate bending of the manifold layer 124 so that the manifold layer 124 closely conforms to the shape of the wound treatment area. The scoring pattern 226 allows the manifold layer 124 to conform to both substantially flat surfaces and curved surfaces at the wound treatment area.

FIG. 6 illustrates a manifold layer 232 according to another embodiment. The manifold layer 232 is generally similar to the manifold layer 124. The manifold layer 232 can be incorporated into the wound dressing 100 as described above with respect to the manifold layer 124. Like numbers are indicated by the same number and parts of the manifold layer 232 are indicated using the prime symbol “′”.

As shown in FIGS. 2-5, the manifold layer 232 is generally symmetrical about the axis B. The manifold layer 232 is substantially T-shaped and includes a first lobe 236, a second lobe 240, and a third lobe 244. The manifold layer 232 can have a length L′ ranging from approximately 7.44 inches to 11.16 inches. In some embodiments, the length L′ is approximately 9.3 inches. The manifold layer 232 can have a width W′ ranging from approximately 7.76 inches to approximately 11.64 inches. In some embodiments, the width W′ is approximately 9.7 inches. The manifold layer 232 can have a thickness T′ ranging from approximately 0.64 inches to 0.96 inches. In some embodiments, the thickness T′ is approximately 0.8 inches.

The manifold layer 232 includes the first lobe 236, the second lobe 240, and the third lobe 244 described above with respect to FIG. 5. The first lobe 236 and the second lobe 240 are substantially elliptical. The first lobe 236 and the second lobe are substantially aligned along the axis A′. A convex portion 248 extends along a portion of the perimeter of the wound dressing 100 that is between the first lobe 236 and the second lobe 240. The convex portion 248 is substantially aligned with the third lobe 244 along the axis B′. The convex portion 248 can have a radius of curvature rc₄ ranging from approximately 4 inches to approximately 6 inches. In some embodiments, the radius of curvature rc₄ can be approximately 5 inches.

### Wound-Interface Layer

The wound-interface layer 128 is shown to include a first surface 222 and a second, wound-facing surface 224 opposite the first surface 222. When the wound dressing 100 is applied to the wound, the first surface 222 faces away from the wound, whereas the second surface 224 faces toward the wound. In some embodiments, the first surface 222 of the wound-interface layer 128 contacts the second surface 224 of the manifold layer 124. In some embodiments, the second surface 224 of the wound-interface layer 128 contacts the patient's tissue. In some embodiments, the wound dressing 100 may not include the wound-interface layer 128.

The wound-interface layer 128 is made of a wicking material that is fluid-permeable and intended to not irritate the patient's tissue. In the illustrated embodiment, the wound-interface layer is a polyester pique-knit fabric, such as Milliken Fabric. In other embodiments, other permeable and non-irritating fabrics can be used. The wound-interface layer 128 can also be treated with antimicrobial materials. In the illustrated embodiment, the wound-interface layer 128 includes silver ions as an antimicrobial material. Other anti-microbial materials may be used in other embodiments.

### Integrated Immobilization Device and NPWT System

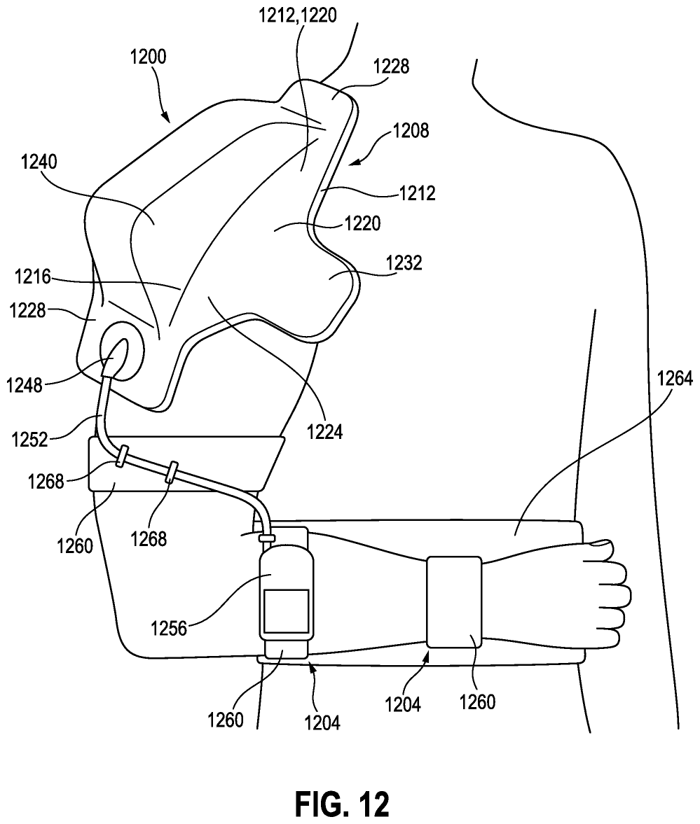

Referring now to FIG. 7, the wound dressing 100 is used in conjunction with an immobilization device 252. The immobilization device 252 is configured to immobilize a patient's arm relative to the patient's shoulder to restrict movement of the patient's shoulder. In the embodiment illustrated in FIG. 7, the immobilization device 252 is a sling. The sling includes an arm-receiving portion 256 and a shoulder strap 260. The arm-receiving portion 256 is configured to receive at least the forearm and elbow of the arm corresponding to the injured shoulder. The shoulder strap 260 is coupled to the arm-receiving portion 256 and is configured to be positioned over a patient's uninjured shoulder to support the arm corresponding to the patient's injured shoulder. In some embodiments, the sling includes a belt (not shown) configured to immobilize the arm-receiving portion 256 of the sling relative to the patient's torso. In other embodiments, the immobilization device 252 can be a belt as illustrated below in FIG. 12.

The NPWT system 262 further includes a removed fluid container 264 and a negative pressure source or pump 268 that are in fluid communication with the wound dressing 100 via the negative pressure conduit 272. In some embodiments, the pump 268 can be a powered pump 268. In such an embodiment, the NPWT system 262 further includes a battery configured to power the pump 268. In other embodiments, the pump 268 is an unpowered pump. In such an embodiment, the pump 268 can be hand-actuated by the patient. The removed fluid container 264 can be configured to store a fluid removed from the incisions 14, 18, 22 (FIG. 1). Removed fluid can include, for example, wound exudate (e.g., bodily fluids), air, fluid that was injected into the wound treatment area during surgery, or any other type of fluid which can be removed from the incision 240 during wound treatment.

The NPWT system 262 is coupled to the wound dressing 100 by the negative pressure conduit 272. The negative pressure conduit 272 has a first end 276 coupled to the reduced-pressure interface 192 of the wound dressing 100 and a second end 280 coupled to the NPWT system 262. In the illustrated embodiment, the negative pressure conduit 272 is a multi-lumen conduit. The negative pressure conduit 272 includes a first lumen 284 and a second lumen 288. The first lumen 284 is configured to apply negative pressure to the wound dressing 100 and to draw exudate into the removed fluid container 264. The second lumen 288 is configured for sensing the pressure of the wound dressing 100. One such NPWT system 262 including a multi-lumen conduit is the SensaT.R.A.C.™ system that is available from Kinetic Concepts, Inc. (KCI) of San Antonio, Tex.

Returning to FIG. 7, the NPWT system 262 is integrated with the immobilization device 252. As illustrated in FIG. 7, the NPWT system 262 is secured within the arm-receiving portion 256 of the immobilization device 252. For example, the NPWT system 262 can be positioned within a pocket of the arm-receiving portion 256, sewn into the arm-receiving portion 256, secured within the arm-receiving portion 256 of the immobilization device 252 using a detachable adhesive such as Velcro, etc. As illustrated in FIG. 7, a portion of the negative pressure conduit 272 proximate the NPWT system 262 is integrated with the arm-receiving portion 256. For example, the negative pressure conduit 272 can be positioned within a passageway of the arm-receiving portion 256, secured within the arm-receiving portion 256 of the immobilization device 252 using a detachable adhesive such as Velcro, etc. Integration of the NPWT system 262 within the immobilization device 252 allows the patient to conveniently transport the NPWT system 262 while the patient is undergoing NPWT.

### Deployment of the Dressing

FIG. 7 illustrates the wound dressing 100 deployed at a representative illustration of a patient's torso. The patient's arm proximate the wounded shoulder is immobilized relative to the patient's torso by the immobilization device 252 to immobilize the shoulder joint. While wound dressing 100 is shown in FIG. 7, the manifold layer 232 can be deployed in a similar manner Referring briefly to FIG. 1, the wound treatment area includes the first incision 14 and surrounding healthy tissue at a front of the patient's shoulder, the second incision 18 and surrounding healthy tissue at a back of the patient's shoulder, and the third incision 22 and surrounding healthy tissue at a top of the patient's arm. As illustrated in FIG. 7, the wound dressing 100 does not over the patient's armpit. As is apparent from comparison of FIGS. 1 and 7, the wound dressing 100 is sized to cover the surface including the entire wound treatment area. A further advantage of covering the entire wound area is that the wound dressing 100 can provide NPWT to the whole wound treatment area to generate negative pressure and lifting forces over the wound treatment area to facilitate wound healing and to facilitate absorption of the fluid injected during surgery by the lymphatic system. In some embodiments, the wound dressing 100 can be used with topically applied pharmaceutical compounds. For example, the wound dressing 100 can be used in conjunction with a silicone gel applied proximate the first incision 14, the second incision 18, and the third incision 22. The silicone gel can reduce scarring at or near the incisions 14, 18, 22.

As illustrated in FIG. 7, the reduced-pressure interface 192 is positioned over the third lobe 212. In other embodiments, the reduced-pressure interface 192 can be positioned elsewhere on the drape layer 120 of the wound dressing 100. The negative pressure conduit 272 extends from the reduced-pressure interface 192 and extends along the patient's arm and into the immobilization device 252. As illustrated using phantom lines, the negative pressure conduit 272 is coupled to the NPWT system 262 integrated with the immobilization device 252. Due to the symmetric shape of the wound dressing 100, the wound dressing can be used to treat wounds in both the left shoulder and the right shoulder.

To deploy the wound dressing 100 to treat a wound treatment area at a shoulder of the patient, a healthcare practitioner removes the cover sheet 148 from the wound dressing 100. The healthcare practitioner then orients the wound dressing 100 relative to the patient's shoulder such that the first lobe 104 overlies an incision and surrounding healthy tissue at a front of a patient's shoulder and the second lobe 108 overlies an incision and healthy tissue at a back of the patient's shoulder. The healthcare practitioner then orients the wound dressing 100 such that the third lobe 112 overlies an incision and surrounding healthy tissue at an upper portion the patient's arm proximate the wounded shoulder. The healthcare practitioner then applies pressure around the perimeter of the margin 160 of the drape layer 120 to secure the second adhesive layer 140 to the patient's tissue. The healthcare practitioner then immobilizes the patient's arm relative to the patient's torso using the immobilization device 252. The healthcare practitioner then inserts the negative pressure conduit 272 into the immobilization device 252 and couples the negative pressure conduit 272 to the NPWT system 262. The healthcare practitioner then actuates the NPWT system 262 to apply negative pressure to the wound treatment area.

### Wound Dressing

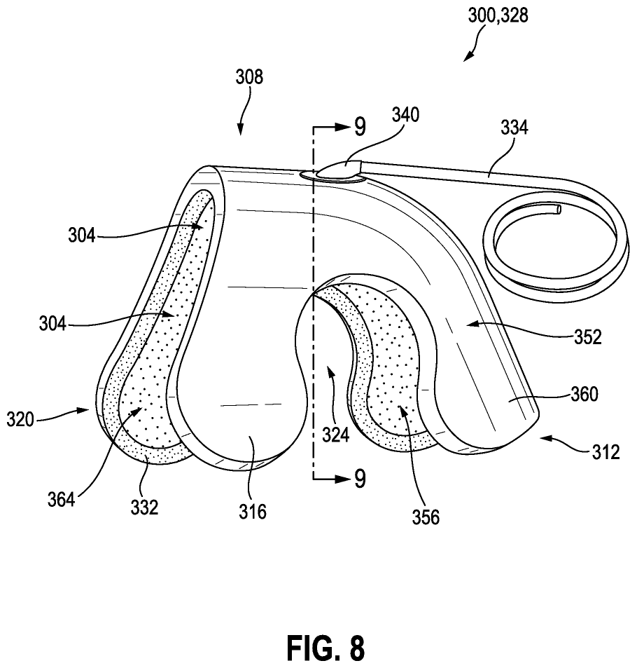

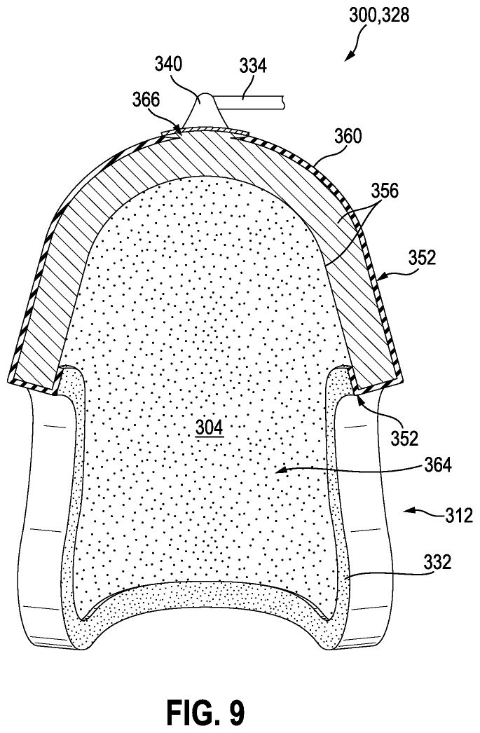

Referring now to FIGS. 8-9, a wound dressing 300 is shown, according to an exemplary embodiment. FIG. 8 illustrates a perspective view of the wound dressing 300 according to an exemplary embodiment. FIG. 9 is a section view of the wound dressing 300 taken along lines 9-9 of FIG. 8.

In various embodiments, the wound dressing 300 can be formed as a substantially elbow-shaped channel 304 that conforms to the three-dimensional shape of a wound treatment area at a shoulder of the patient. The elbow-shaped channel 304 includes a first portion 308 and a second portion 312 that is angled relative to the first portion 308. In some embodiments, the second portion 312 is at an obtuse angle relative to the first portion 308. A first lobe 316 and a second portion 312 extend from the first portion 308. The first lobe 316 and the second lobe 320 are generally perpendicular to the first portion 308. The first portion 308 is configured to overlie a top portion of a patient's shoulder. The first lobe 316 is configured to overlie an incision and healthy tissue surrounding the incision at a front portion of the patient's shoulder. The second lobe 320 is configured to overlie an incision and healthy tissue surrounding the incision at a back part of the patient's shoulder. The second portion 312 is configured to overlie an incision and healthy tissue surrounding the incision at a top of the patient's arm proximate the wounded shoulder. In some embodiments, the second portion 312 defines a third lobe. A channel 324 extends between the first lobe 316 and the second portion 312 and the second lobe 320 and the second portion 312 such that the wound dressing 300 does not cover the patient's armpit.

The wound dressing 300 includes the manifold layer 328, and an adhesive layer 332. The wound dressing 300 further includes a reduced-pressure interface 340 configured to engage the negative pressure conduit 334 of a NPWT system 348.

### Manifold Layer

Referring to FIGS. 8 and 9, the manifold layer 328 includes an exterior surface or skin 352 and an interior portion 356. The skin 352 is a fluid-impermeable skin that surrounds the interior portion 356. The interior portion 356 is made from a porous and permeable foam-like material, and, more particularly, a reticulated, open-cell polyurethane or polyether foam that allows good permeability of wound fluids while under a reduced pressure. One such foam material that has been used is the VAC® Granufoam™ material that is available from Kinetic Concepts, Inc. (KCI) of San Antonio, Tex. Any material or combination of materials might be used for the manifold layer 328 provided that the manifold layer 328 is operable to distribute the reduced pressure and provide a distributed compressive force along the wound treatment area.

Referring to FIGS. 8 and 9, the manifold layer 328 includes a first surface 360 and a second, wound-facing surface 364 opposite the first surface 360. When the wound dressing 300 is applied to a wound, the first surface 360 faces away from the wound, whereas the second surface 364 faces toward the wound. As illustrated in FIG. 8, the skin 352 extends over the first surface 360 of the manifold layer 328. The reduced-pressure interface 340 is coupled to the first surface 360 of the manifold layer 328. As is best shown in FIG. 9, a portion 366 of the skin 352 is removed from the first surface 360 of the manifold layer 328 to expose the interior portion 356. The reduced-pressure interface 340 is coupled to the first surface 360 of the manifold layer 328 generally over the portion of the first surface 360 of the manifold layer 328 that does not include the skin 352 so that the reduced-pressure interface 340 is in fluid communication with the interior portion 356 of the second surface 364 and therefore, with the wound treatment area. The reduced-pressure interface 340 is secured to the first surface 360 in a substantially fluid-tight seal.

The second surface 364 defines surface of the elbow-shaped channel 304. The skin 352 has been removed from the second surface 364 generally inward (e.g. towards a center) of the perimeter of the second surface 364 to expose the interior portion 356 (e.g., the open-cell foam) of the manifold layer 328. Accordingly, the skin 352 extends about a perimeter of the second surface 364 and the foam layer extends across the second surface 364 inward of the perimeter. As best illustrated in FIG. 9, the adhesive layer 332 is secured to the skin 352 extending about the perimeter of the second surface 364. The adhesive layer 332 can be an acrylic adhesive, a silicone adhesive, and/or other adhesives. Accordingly, the manifold layer 328 can be secured to a patient's shoulder in a substantially fluid-tight seal to provide a sealed space over the wound treatment area without requiring the drape layer.

### Method of Manufacturing the Manifold Layer

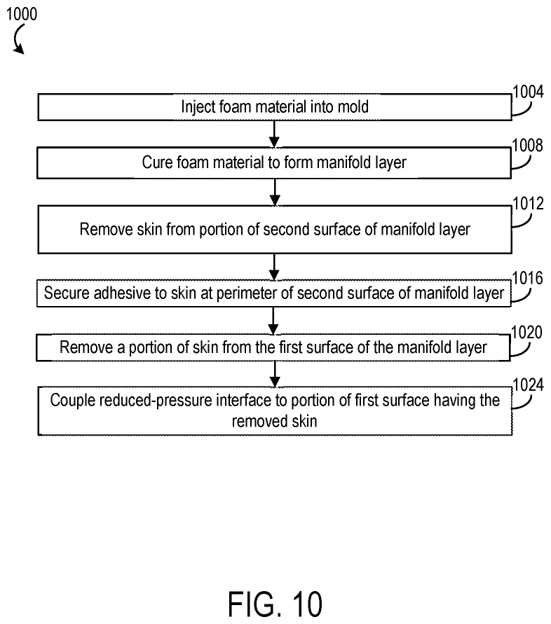

Referring now to FIG. 10, a method 1000 for manufacturing the manifold layer 328 is shown, according to an example embodiment. At step 1004, a foam material is injected into a mold defining a substantially elbow-shaped channel having a first portion and a second portion angled relative to the first portion. In some embodiments, the second portion is at a substantially obtuse angle relative to the first portion. The first portion further includes a first lobe and a second lobe extending from and generally perpendicular to the first portion. The foam material can include any porous and permeable foam-like material, and, more particularly, a reticulated, open-cell polyurethane or polyether foam that allows good permeability of wound fluids while under a reduced pressure. One such foam material that has been used is the VAC® Granufoam™ material that is available from Kinetic Concepts, Inc. (KCI) of San Antonio, Tex. Any material or combination of materials might be used for the manifold layer 328 provided that the manifold layer 328 is operable to distribute the reduced pressure and provide a distributed compressive force along the wound treatment area.

At step 1008, the foam material is cured so that a substantially fluid-impermeable skin 352 forms over an exterior surface of the manifold layer 328 material and an open-cell foam forms in an interior portion 356 of the manifold layer 328 material. At step 1012, a portion of the skin 352 is removed from the second surface 364 of the manifold layer 328 inward of the perimeter of the second surface 364 to expose the foam of the interior portion 356. In some embodiments, a patient-contacting layer is secured to the exposed foam of the interior portion 356 of the manifold layer 328. At step 1016, an adhesive layer 332 is secured to the skin 352 at the perimeter of the second surface 364. In some embodiments, a backing layer can be engaged with the adhesive layer 332. At step 1020, the portion 366 of the skin 352 of the first surface 360 of the manifold layer 328 is removed. At step 1024, a reduced pressure interface coupled to the first surface 360 of the manifold layer 328. The reduced-pressure interface 340 substantially overlies the portion 366 of the first surface 360 without the skin 352. The reduced-pressure interface 340 is in fluid communication with the second surface 364 of the manifold layer 328 and the wound treatment area through the portion 336 of the first surface 360 without the skin 352. The reduced-pressure interface 340 is sealed to the first surface 360 of the manifold layer 328 to form a substantially fluid-tight connection.

### Deployment of the Wound Dressing

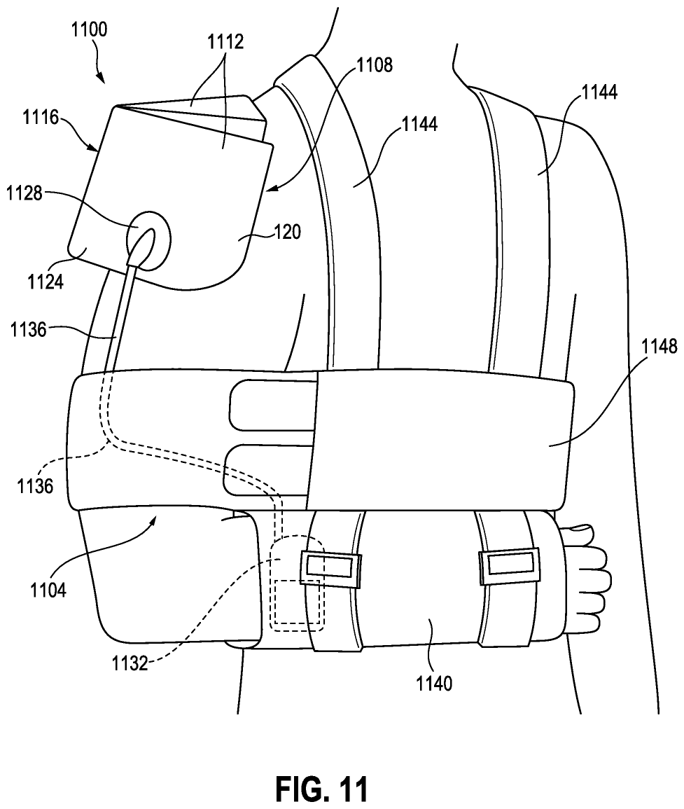

FIGS. 11-14 illustrate exemplary wound dressings 1100, 1200, 1300 deployed on a wound treatment area of a shoulder of a representative patient. The exemplary wound dressings 1100, 1200, 1300 are substantially similar to the wound dressing 300 discussed above with respect to FIGS. 8 and 9. Exemplary immobilization devices 1104, 1204, 1304 immobilize the arm corresponding to the wounded shoulder relative to a torso of the representative patient.

Referring now to FIG. 11, a wound dressing 1100 and an immobilization device 1104 are shown, according to an exemplary embodiment. The wound dressing 1100 defines a bowl-shaped channel 1108 that includes a first portion 1112 that is angled relative to a second portion 1116. In some embodiments, the second portion 1116 is oriented at an obtuse angle relative to the first portion 1112. The wound dressing 1100 is shaped to conform to the three-dimensional area of a wound treatment area on the patient's shoulder. As illustrated in FIG. 11, The wound dressing 1100 is deployed on the patient such that the first portion 1112 covers an incision and surrounding healthy tissue at a front of the patient's shoulder, a top portion of the patient's shoulder, and an incision and surrounding healthy tissue at a back of the patient's shoulder. The first portion 1112 includes a first lobe 1120 and a second lobe 1124. The first lobe 1120 is configured to cover the incision and surrounding healthy tissue at the front of the patient's shoulder. The second lobe 1124 is configured to cover the incision and surrounding healthy tissue at the back of the patient's shoulder. The second portion 1116 of the wound dressing 1100 covers an incision and surrounding tissue at a top portion of the patient's arm. The adhesive layer (not shown) secures the wound dressing 1100 to the patient's tissue in a substantially fluid-tight seal.

With continued reference to FIG. 11, the second portion 1116 of the wound dressing 1100 includes a reduced-pressure interface 1128. In other embodiments, the reduced-pressure interface 1128 may be positioned elsewhere on the wound dressing 1100. For example, in some embodiments, the reduced-pressure interface 1128 can be positioned on the first portion 1112 of the wound dressing 1100. The reduced-pressure interface 1128 is coupled to a negative pressure conduit 1136 that is in fluid communication with a NPWT system 1132. The negative pressure conduit 1136 is substantially similar to the negative pressure conduit 334 and is not disclosed in greater detail herein. The NPWT system 1132 is substantially similar to the NPWT system 262 discussed above. Due to the symmetry of the wound dressing 1100, the wound dressing can be deployed on a patient's right shoulder or a patient's left shoulder without requiring modification.

In the illustrated embodiment, the immobilization device 1104 is a sling. In other embodiments, the immobilization device 1104 can be a belt, such as the belt 1204 illustrated in FIG. 12. The immobilization device 1104 includes an arm-receiving portion 1140, shoulder straps 1144, and a belt 1148. The arm-receiving portion 1140 is configured to receive around the arm of the patient that corresponds to the wounded shoulder. The shoulder straps 1144 are connected to the arm-receiving portion 1140 and are positionable over the patient's shoulders to support the patient's arm. As shown in FIG. 11, the shoulder strap 1144 positioned proximate the wounded shoulder is positioned inward of the wound treatment area such that the shoulder strap 1144 does not interfere with the wound dressing 1100. The belt 1148 is configured to wrap around the patient's torso to immobilize the patient's arm relative to the patient's torso to immobilize the wounded shoulder. In some embodiments, the belt 1148 is coupled to the shoulder straps 1144. In other embodiments, the belt 1148 is positioned over the shoulder straps 1144 such that the shoulder straps 1144 are positioned between the belt 1148 and the patient's torso.

The NPWT system 1132 and a portion of the negative pressure conduit 1136 are integrated with the immobilization device 1104. The NPWT system 1132 can be secured within arm-receiving portion 1140 or the belt 1148 of the immobilization device 1104. For example, the NPWT system 1132 can be positioned within a pocket of the arm-receiving portion 1140 or the belt 1148, sewn into arm-receiving portion 1140 or the belt 1148, secured within the arm-receiving portion 1140 or secured within or to the belt 1148 of the immobilization device 1104 using a detachable adhesive such as Velcro, etc. A portion of the negative pressure conduit 1136 proximate the NPWT system 1132 is integrated the belt 1148 and/or the arm-receiving portion 1140. For example, the negative pressure conduit 1136 extends from the wound dressing 1100 and along the arm of the patient. The negative pressure conduit 1136 then extend through loops or clips secured to the arm-receiving portion 1140 and/or the belt 1148, is secured to the arm-receiving portion 1140 or the belt 1148 using a detachable adhesive such as Velcro, extends through one or more passages in the arm-receiving portion 1140 or the belt 1148, etc.

Referring now to FIG. 12, a wound dressing 1200 and an immobilization device 1204 are shown, according to an exemplary embodiment. The wound dressing 1200 defines a bowl-shaped channel 1208 that includes a first portion 1212 that is angled relative to a second portion 1216. In some embodiments, the second portion 1216 is oriented at an obtuse angle relative to the first portion 1212. The wound dressing 1200 is shaped to conform to the three-dimensional area of a wound treatment area on the patient's shoulder. As illustrated in FIG. 11, the wound dressing 1200 is deployed on the patient such that the first portion 1212 covers an incision and surrounding healthy tissue at a front of the patient's shoulder, a top portion of the patient's shoulder, and an incision and surrounding healthy tissue at a back of the patient's shoulder. The first portion 1212 includes a first lobe 1220 and a second lobe 1224. The first lobe 1220 is configured to cover the incision and surrounding healthy tissue at the front of the patient's shoulder. The second lobe 1224 is configured to cover the incision and surrounding healthy tissue at the back of the patient's shoulder. The second portion 1216 of the wound dressing 1200 covers an incision and surrounding tissue at a top portion of the patient's arm. The adhesive layer (not shown) secures the wound dressing 1200 to the patient's tissue in a substantially fluid-tight seal.

As shown in FIG. 12, a flange 1228 extends around a perimeter of the bowl-shaped channel 1208. A first tab 1232 and a second tab (not shown) extend from the flange 1228 at or proximate a midpoint of the bowl-shaped channel 1208. The first tab 1232, the second tab, and the flange 1228 can increase a surface area at and extending from the perimeter of the wound dressing 1200 to increase an amount of adhesive that can be used to secure the wound dressing 1200 to the patient's tissue. In some embodiments, the first tab 1232, the second tab, and the flange 1228 are integrally formed with the bowl-shaped channel 1208. In other embodiments, the first tab 1232, the second tab, and the flange 1228 can be made separately from the bowl-shaped channel 1208 and secured to the bowl-shaped channel 1208.

As shown in FIG. 12, a ridge 1240 of foam extends above the bowl-shaped channel 1208. The ridge 1240 increases an amount of foam that can be compressed by the negative pressure source, which in turn results in greater lifting forces generated around the wound treatment area. The greater lifting forces can increase an amount of the fluid injected during surgery that is absorbed by the patient's lymphatic system.

With continued reference to FIG. 12, the second portion 1216 of the wound dressing 1200 includes a reduced-pressure interface 1248. In other embodiments, the reduced-pressure interface 1248 may be positioned elsewhere on the wound dressing 1200. For example, in some embodiments, the reduced-pressure interface 1248 can be positioned on the first portion 1212 of the wound dressing 1200. The reduced-pressure interface 1248 is coupled to a negative pressure conduit 1252 that is in fluid communication with a NPWT system 1256. The negative pressure conduit 1252 is substantially similar to the negative pressure conduit 334 and is not disclosed in greater detail herein. The NPWT system 1256 is substantially similar to the NPWT system 334 discussed above. Due to the symmetry of the wound dressing 1200, the wound dressing can be deployed on a patient's right shoulder or a patient's left shoulder without requiring modification.

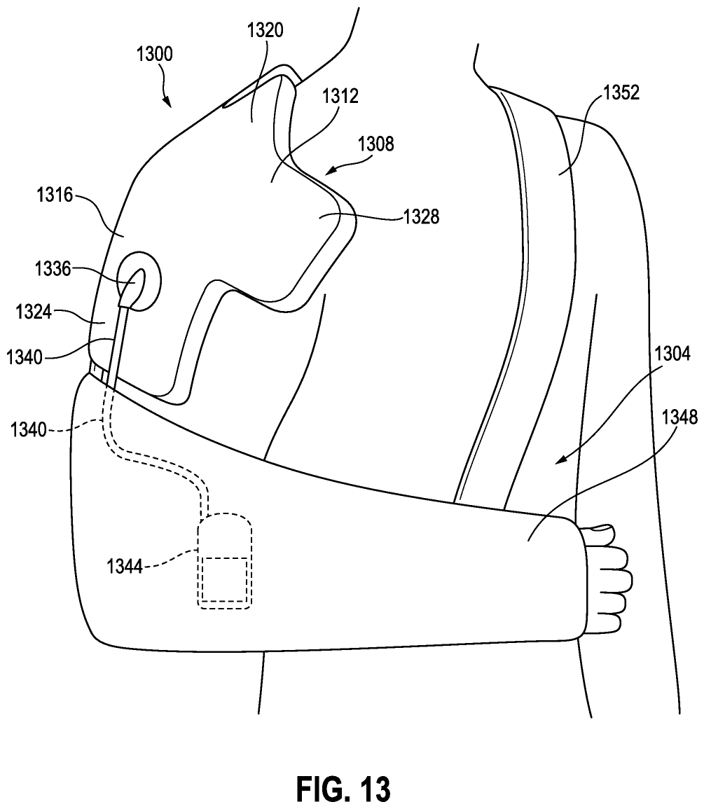

With continued reference to FIG. 12, the immobilization device 1204 of the illustrated embodiment is a belt. In other embodiments, the immobilization device 1204 can be a sling, such as the slings 1104, 1304 illustrated in FIGS. 11 and 13, respectively. The immobilization device 1204 includes an arm-receiving portion 1260 and a belt 1264. In the illustrated embodiment, the arm-receiving portion 1260 includes a plurality of straps that are configured to wrap around the arm of the patient that corresponds to the wounded shoulder. The arm-receiving portion 1260 is connected to the belt 1264, together with the belt 1264 immobilize the patient's arm relative to the patient's torso to immobilize the wounded shoulder. The NPWT system 1256 and at least a portion of the negative pressure conduit 1252 are integrated with the immobilization device 1204. As illustrated in FIG. 12, the NPWT system 1256 is secured within the belt 1264 of the immobilization device 1204. For example, the NPWT system 1256 can be positioned within a pocket of the belt 1264, sewn into the belt 1264, secured within or to belt 1264 of the immobilization device 1204 using a detachable adhesive such as Velcro, etc. A portion of the negative pressure conduit 1252 proximate the NPWT system 1256 is integrated with the arm-receiving portion 1260. For example, the negative pressure conduit 1252 extend through loops or clips 1268 secured to the arm-receiving portion 1260, be secured to the arm-receiving portion 1260 using a detachable adhesive such as Velcro, etc.

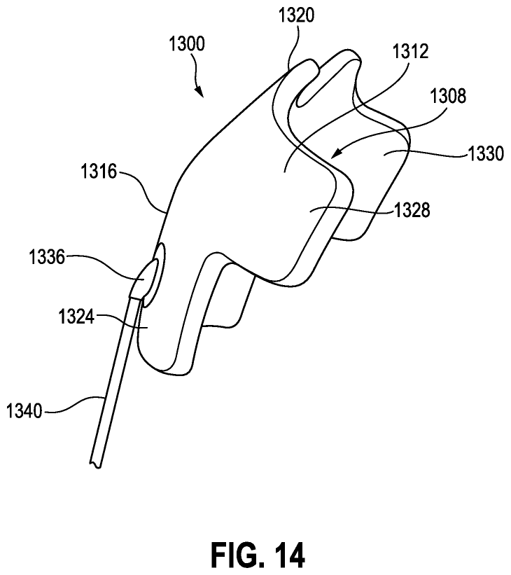

FIG. 13 illustrates a wound dressing 1300 and an immobilization device 1304 are shown, according to an exemplary embodiment. FIG. 14 illustrates a perspective view of the wound dressing 1300 according to an exemplary embodiment. The wound dressing 1300 defines a bowl-shaped channel 1308 (FIG. 14) that includes a first portion 1312 that is angled relative to a second portion 1316. In some embodiments, the second portion 1316 is oriented at an obtuse angle relative to the first portion 1312. The wound dressing 1300 is shaped to conform to the three-dimensional area of a wound treatment area on the patient's shoulder. As illustrated in FIG. 13, The wound dressing 1300 is deployed on the patient such that the first portion 1312 covers an incision and surrounding healthy tissue at a front of the patient's shoulder, a top portion of the patient's shoulder, and an incision and surrounding healthy tissue at a back of the patient's shoulder. The first portion 1312 includes a first lobe 1320 and a second lobe 1324. The first lobe 1320 is configured to cover the incision and surrounding healthy tissue at the front of the patient's shoulder. The second lobe 1324 is configured to cover the incision and surrounding healthy tissue at the back of the patient's shoulder. The second portion 1316 of the wound dressing 1300 covers an incision and surrounding tissue at a top portion of the patient's arm. The adhesive layer (not shown) secures the wound dressing 1300 to the patient's tissue in a substantially fluid-tight seal.

As shown in FIGS. 13 and 14, a first tab 1328 and a second tab 1330 extend from the bowl-shaped channel 1308 at or proximate a midpoint of the bowl-shaped channel 1308. The first tab 1328 and the second tab 1330 increase a surface area at and extending from the perimeter of the wound dressing 1300 to increase an amount of adhesive that can be used to secure the wound dressing 1300 to the patient's tissue. In some embodiments, the first tab 1328 and the second tab 1330 are integrally formed with the bowl-shaped channel 1308. In other embodiments, the first tab 1328 and the second tab 1330 can be made separately from the bowl-shaped channel 1308 and secured to the bowl-shaped channel 1308.

With continued reference to FIGS. 13 and 14, the second portion 1316 of the wound dressing 1300 includes a reduced-pressure interface 1336. In other embodiments, the reduced-pressure interface 1336 may be positioned elsewhere on the wound dressing 1300. For example, in some embodiments, the reduced-pressure interface 1336 can be positioned on the first portion 1312 of the wound dressing 1300. The reduced-pressure interface 1336 is coupled to a negative pressure conduit 1340 that is in fluid communication with a NPWT system 1344. The negative pressure conduit 1340 is substantially similar to the negative pressure conduit 334 and is not disclosed in greater detail herein. The NPWT system 1344 is substantially similar to the NPWT system 334 discussed above. Due to the symmetry of the wound dressing 1300, the wound dressing can be deployed on a patient's right shoulder or a patient's left shoulder without requiring modification.

The NPWT system 1344 is integrated with the immobilization device 1304. In the illustrated embodiment, the immobilization device 1304 is a sling. In other embodiments, the immobilization device 1304 can be a belt, such as the belt 1208 illustrated in FIG. 12. The immobilization device 1304 includes an arm-receiving portion 1348 and a shoulder strap 1352. In some embodiments, the immobilization device 1304 further includes a belt. The arm-receiving portion 1348 is configured to receive an arm of the patient that corresponds to the wounded shoulder. The shoulder strap 1352 is configured to engage the uninjured shoulder to support the arm in the arm-receiving portion. As illustrated in FIG. 13, the NPWT system 1344 is secured within the arm-receiving portion 1348 of the immobilization device 1304. For example, the NPWT system 1344 can be positioned within a pocket of the arm-receiving portion 1348, sewn into the arm-receiving portion 1348, secured within the arm-receiving portion 1348 of the immobilization device 1304 using a detachable adhesive such as Velcro, etc. As illustrated in FIG. 13 a portion of the negative pressure conduit 1340 proximate the NPWT system 1344 is integrated with the arm-receiving portion. For example, the negative pressure conduit 1340 can be positioned within a passageway of the arm-receiving portion 1348, secured within the arm-receiving portion 1348 of the immobilization device 1304 using a detachable adhesive such as Velcro, etc.

To deploy the wound dressings 1100, 1200, 1300 to treat a wound treatment area at a shoulder of the patient, a healthcare practitioner removes the cover sheet the adhesive layer (not shown). The healthcare practitioner then orients the wound dressing 1100, 1200, 1300 relative to the patient's shoulder such that the first portion 1112, 1212, 1312 overlies an incision and surrounding healthy tissue at a front of a patient's shoulder, a top of the patient's shoulder, and an incision and healthy tissue at a back of the patient's shoulder. More specifically, the first lobe 1120, 1220, 1320 is positioned over the incision and surrounding healthy tissue at the front of the patient's shoulder and the second lobe 1124, 1224, 1324 is positioned over the incision and surrounding healthy tissue at the back of the patient's shoulder. The healthcare practitioner then orients the wound dressing 1100, 1200, 1300 such that the second portion 1116, 1216, 1316 overlies an incision and surrounding healthy tissue at an upper portion of an arm of the patient corresponding to the wounded shoulder. The healthcare practitioner then applies pressure around the perimeter of the wound dressing 1100, 1200, 1300 to secure the adhesive layer (not shown) to the patient's tissue. The healthcare practitioner then immobilizes the patient's arm relative to the patient's torso using the immobilization device 1104, 1204, 1304. The healthcare practitioner then couples the negative pressure conduit 1136, 1252, 1340 of the wound dressing 1100, 1200, 1300 to the NPWT system 1132, 1256, 1344. The healthcare practitioner then actuates the NPWT system 1132, 1256, 1344 to apply negative pressure to the wound treatment area.

### Configuration of Exemplary Embodiments

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements can be reversed or otherwise varied and the nature or number of discrete elements or positions can be altered or varied. Accordingly, all such modifications are intended to be included within the scope of the present disclosure. The order or sequence of any process or method steps can be varied or re-sequenced according to alternative embodiments. Other substitutions, modifications, changes, and omissions can be made in the design, operating conditions and arrangement of the exemplary embodiments without departing from the scope of the present disclosure.

What is claimed is:

1. A negative pressure wound treatment (NPWT) dressing system for use with shoulder incisions, the wound dressing system, comprising:
a wound dressing comprising:
a drape layer having a first surface and a second, wound-facing, surface, wherein the drape layer is substantially impermeable to liquid and substantially permeable to vapor;
a manifold layer having a first surface and a second, wound-facing surface, the manifold layer having a first lobe, a second lobe substantially aligned with the first lobe, and a third lobe extending substantially perpendicular to the first lobe and the second lobe; and
a reduced-pressure interface integrated with the drape layer;
an immobilization device configured to immobilize a shoulder of a patient; and
a negative pressure source in fluid communication with the reduced pressure interface, the negative pressure source coupled to the immobilization device.

2. The NPWT dressing system of claim 1, wherein the first lobe is configured to overlie a wound treatment area on a front portion of a patient's shoulder, the second lobe is configured to overlie a wound treatment area on a back portion of the patient's shoulder, and the third lobe is configured to overlie treatment area on a top portion of the patient's arm.

3. The NPWT dressing system of claim 1, wherein the negative pressure source is coupled to the reduced-pressure interface by a multi-lumen conduit.

4. The NPWT dressing system of claim 1, wherein the first lobe and the second lobe are substantially half-ellipses and the third lobe is substantially circular.

5. The NPWT dressing system of claim 1, wherein the wound dressing further comprises a drain interface and the wound dressing system further comprises a removed fluid reservoir in fluid communication with the drain interface and coupled to the immobilization device.

6. The NPWT dressing of claim 1, wherein the wound dressing further comprises an adhesive layer configured to secure the drape layer to the manifold layer and configured to secure the wound dressing to a patient's tissue.

7. A negative pressure wound therapy (NPWT) dressing, comprising:
a drape layer having a first surface and a second, wound-facing, surface, wherein the drape layer is substantially impermeable to liquid and substantially permeable to vapor;
a manifold layer having a first surface and a second, wound-facing surface, the manifold layer having a first lobe, a second lobe generally aligned with the first lobe, and a third lobe extending substantially perpendicular to the first lobe and the second lobe;
a base layer configured to secure the drape layer to the manifold layer, and configured to secure the wound dressing to a patient's tissue; and
a reduced-pressure interface integrated with the drape layer.

8. The NPWT dressing of claim 7, wherein the first lobe is configured to overlie a wound treatment area on a front portion of a patient's shoulder, the second lobe is configured to overlie a wound treatment area on a back portion of the patient's shoulder, and the third lobe is configured to overlie treatment area on a top portion of the patient's arm.

9. The NPWT dressing of claim 7, wherein the manifold layer is substantially T-shaped.

10. The NPWT dressing of claim 7, wherein the first lobe and the second lobe are substantially half-ellipses and the third lobe is substantially circular.

11. The NPWT dressing of claim 10, wherein the third lobe is connected to the first lobe and the second lobe by a connection portion having a width smaller than a diameter of the third lobe.

12. The NPWT dressing of claim 7, wherein a concave portion is positioned between the first lobe and the second lobe and substantially opposite the third lobe.

13. The NPWT dressing of claim 7, wherein the manifold layer includes a scoring pattern formed therein, and the manifold layer is configured to bend about at least one of the scores of the scoring pattern.

14. The NPWT dressing of claim 7, wherein a radius of curvature of the first lobe and the second lobe is between substantially 1.4 inches and substantially 1.56 inches.

15. The NPWT dressing of claim 7, wherein a radius of curvature of the third lobe is substantially 1.6 inches-2.4 inches.

16. A negative pressure wound treatment (NPWT) dressing system for use with shoulder incisions, the wound dressing system, comprising:
a wound dressing comprising a manifold layer defining a substantially elbow-shaped channel having a first portion configured to receive an upper portion of a shoulder of a patient and a second portion angled relative to the first potion and configured to receive an upper portion an arm of the patient, the manifold layer having a first surface that is substantially impermeable to fluid and a second, wound-facing surface that is substantially permeable to fluid;
an adhesive layer coupled along a perimeter of the second surface of the manifold layer and configured to secure the wound dressing to the patient's tissue;
a reduced pressure interface integrated with the first surface of the manifold layer;
an immobilization device configured to immobilize the shoulder; and
a negative pressure source in fluid communication with the reduced pressure device, the negative pressure source coupled to the immobilization device.

17. The NPWT dressing of claim 16, wherein the second portion is at a substantially obtuse angle relative to the first portion.

18. The NPWT dressing of claim 16, wherein first portion of the manifold layer includes a first lobe configured to overlie a front portion of the patient's shoulder and a second lobe configured to overlie a back portion of the patient's shoulder.

19. The NPWT dressing of claim 16, wherein the first lobe and the second portion define a first opening therebetween and the second lobe and the second portion define a second opening therebetween, the first opening and the second opening configured to facilitate bending of the manifold layer.

20. The NPWT dressing of claim 16, wherein a portion of the first surface of the manifold layer adjacent the reduced pressure interface has been removed to provide fluid communication between the negative pressure source and the second surface of the wound dressing.

21. A method of forming a three-dimensional wound-dressing shaped to receive a shoulder, the method comprising:
injection-molding a foam into a mold defining a substantially elbow-shaped channel having a first surface and a second, wound-facing, surface that is substantially permeable to fluid;
curing the foam such that an exterior layer of the foam is fluid-impermeable and an interior portion of the foam is porous;
removing at least a portion of the exterior layer of the foam from the second surface substantially inward of a perimeter of the second surface;
securing an adhesive layer to the perimeter of the second surface, the adhesive layer configured to secure the wound dressing to a patient's tissue;
removing a portion of the exterior layer of the foam from the first surface; and
positioning a reduced-pressure interface over the removed portion of the exterior layer of the foam of the first surface.

22. A method of deploying negative pressure wound therapy (NPWT) dressing on a shoulder, the method comprising:
immobilizing a shoulder of a patient relative to a body of the patient with an immobilization device;
securing a wound dressing to a shoulder treatment area, the securing comprising:
securing a first lobe of a wound dressing manifold proximate a wound treatment area on a front of a shoulder of the patient, securing a second lobe of the wound dressing manifold proximate a wound treatment area on a back of the shoulder, and securing a third lobe of the wound dressing proximate a treatment area on a top portion of an arm of the patient, wherein the first lobe is substantially aligned with the second lobe;
coupling the negative pressure source to a reduced-pressure interface of the wound dressing; and
coupling the negative pressure source to the immobilization device.

23. The NPWT dressing system of claim 22, wherein the negative pressure source is coupled to the reduced-pressure interface by a multi-lumen conduit.

24. The NPWT dressing system of claim 22, wherein the third lobe is substantially perpendicular to the first lobe and the second lobe, the first lobe and the second lobe are substantially half-ellipses, and the third lobe is substantially circular.

25. The NPWT dressing of claim 22, wherein the wound dressing defines a substantially elbow-shaped channel including a first portion and a second portion angled relative to the first portion, the first portion including the first lobe and the second lobe and the second portion defining the third lobe.

26. The NPWT dressing system of claim 22, further comprising the step of coupling a removed fluid reservoir to a drain interface of the wound dressing and coupling the removed fluid reservoir to the immobilization device.
